(19) United States
(12) Patent Application Publication
(10) Pub. No.: US 2009/0069703 A1
(43) Pub. Date: Mar. 12, 2009

(54) SYSTEM FOR ARTIFACT DETECTION AND ELIMINATION IN AN ELECTROCARDIOGRAM SIGNAL RECORDED FROM A PATIENT MONITOR

(75) Inventors: George Takla, Strongsville, OH (US); Kenneth A. Loparo, Chesterland, OH (US); Bala Nair, Bellevue, WA (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignees: The Cleveland Clinic Foundation; Case Western Reserve University

(21) Appl. No.: 12/116,235

(22) Filed: May 7, 2008

Related U.S. Application Data
(60) Provisional application No. 60/930,557, filed on May 17, 2007.

(51) Int. Cl. A61B 5/0402

(52) U.S. Cl. 600/509

(57) ABSTRACT

A system eliminates artifacts from an electrocardiogram signal. The system includes a monitor for receiving an electrocardiogram signal from a patient and a microprocessor utilizing a shift-invariant wavelet transform for decomposing the electrocardiogram signal into a plurality of scales. The microprocessor applies rules to the scales for removing artifacts from the scales. The microprocessor reassembles the plurality of scales to produce a reconstructed and accurate electrocardiogram signal without the artifacts.

### RELATED APPLICATION

This application claims priority from U.S. Provisional Application No. 60/930,557, filed May 17, 2007, the subject matter of which is incorporated herein by reference.

### FIELD OF INVENTION

The present invention relates to processing an electrocardiogram signal and, more particularly, to utilizing a transform function to eliminate undesirable artifacts from the electrocardiogram signal.

### BACKGROUND OF THE INVENTION

A conventional electrocardiogram (ECG) signal provides a physician with crucial information on a patient's heart function. The ECG signal is displayed by a patient monitor and used to monitor the hemodynamics of a patient who will undergo or is undergoing a surgical procedure. However, many physiological and environmental artifacts may interfere with the ECG signal being displayed. These artifacts not only may obstruct the correct hemodynamic information being displayed by the monitor, but also could result in erroneous data being displayed.

The ECG signal has been rigorously studied to provide algorithms that may detect patterns in the ECG, such as cardiac rhythms, cardiac arrhythmias, and premature beats. The ECG cardiac rhythms include the P-wave, QRS complex, T-wave, and ST segment. Cardiac arrhythmias, such as atrial fibrillation and ventricular fibrillation, may be of primary interest to clinicians. Additionally, the premature contractions of the atria and ventricle, and junctional blocks, may be of main interest to physicians and researchers. Conventional research has focused on detecting one of the patterns, regardless of the other patterns.

Some of the conventional algorithms have been advantageous for QRS complex detection within a noisy ECG signal. Other conventional algorithms have been advantageous for eliminating artifacts. However, many of these algorithms, while removing certain artifacts, also inadvertently remove useful signal information. For example, P and T waves may be eliminated if an inappropriate filter is used to remove baseline wander in the detection of ST segments. Also, in spite of the advantages of conventional algorithms, artifacts may still be present in the processed ECG signal in different environments.

Conventional algorithms for signal processing have been evaluated to develop a mechanism for detecting and eliminating artifacts from ECG signals acquired in an operating room. ECG signals acquired from the operating room have been processed using four main algorithms: a) feature classification using neural networks; and b) signal separation using independent components analysis; and c) signal decomposition using wavelets. Certain types of wavelets provided a signal decomposition that may be adequately consistent to establish mechanisms for eliminating artifacts, while still maintaining essential features of the ECG signal that are necessary for clinical evaluation of a patient.

Generally, patient monitors, along with associated hardware modules, are used to acquire hemodynamic signals of a patient in an operating room (OR). A hardware module may interface with different types of transducers and sense signals of the electrocardiogram, the arterial blood pressure, and the pulse-oximetry signals. These signals (or waveforms) may be further processed by a patient monitor to derive parameters such as heart rate, blood pressure, and oxygen saturation. The waveforms and parameters may be displayed by the patient monitor to convey vital clinical information to health care providers.

Five electrodes may be attached to different locations on a patient's body to measure electrical signals originating from the electrical activity of the heart. In a conventional 5-electrode configuration, a single electrode is attached to the chest acting as a reference electrode. The potential difference between each of the other four electrodes placed oh both arms and legs, and the reference electrode, is measured to obtain the ECG signal. The four ECG signals may be different in shape and magnitude due to the different locations of the electrodes on the body of the patient. The patient monitor may analyze the ECG signal and primarily extract a heart rate parameter.

The patient monitor may also acquire other signals. These may include an arterial blood pressure signal and a pulse-oximetry signal. The information embedded in these signals may also reflect the heart function similar to the ECG signal. This information may also be used to aid in the process of artifact detection/elimination within the ECG signal.

To measure blood pressure, a catheter may be inserted in an artery, generally in the forearm or hand. The external end of the catheter is attached to a pressure sensor that measures the blood pressure in the artery. The sensed blood pressure signal (waveform) is then sent to the patient monitor. The patient monitor may process the blood pressure waveform to derive the systolic, mean, and diastolic pressures. Also, a pulse rate (same as heart rate for most cases) may be derived from the blood pressure waveform and displayed as a supplemental parameter.

Further, an infrared LED may illuminate a finger of the patient and a light intensity sensor may be placed at the other side of the finger. Changes in blood photoplethysmographic characteristics, due to the blood perfusion level, modifies the infrared light that is sensed. The transducer may measure the infrared intensity during the cardiac cycle and produce a signal representative of oxygenation level. This oxygenation signal, called the pulse-oximetry signal, may be processed to obtain oxygen saturation (SpO2) and pulse rate parameters. Processing of the oxygenation signal may occur in a hardware module interfaced to the patient monitor. The patient monitor may receive and display the oxygenation signal, the SpO2 parameter, and the pulse rate parameter.

A patient monitor may receive digitized signals from the interfacing hardware module. The sampling rates of these signals may vary due to different Nyquist sampling requirements of the signals being acquired. For example, a General Electric patient monitor (SOLAR 9500/TRAM system) samples the ECG signal at 240 Hz, while the arterial and the SpO2 parameters may be sampled at 120 Hz. The patient monitor receives these digitized signals and determines physiological parameters that may be of interest. The parameters and the original digitized Waveform may be displayed to the clinicians on the patient monitors.

In an operating room, artifacts affecting the ECG signal may mainly be caused by electromagnetic interference, movement artifact, electromyographic (EMG) interference, and/or improper application of the ECG electrodes and leads. Other sources of electromagnetic interference are electronic devices used during surgery, such as an electro-surgery knife, a cardiopulmonary bypass machine or an electric warming blanket. Electromagnetic interference caused by these devices may appear as artifact signals within the ECG signal and may be misinterpreted by the patient monitor. ECG artifacts may also be generated by deformations of the skin caused by patient movement or shivering, which may change the impedance and capacitance of the skin around an ECG electrode. The impedance and capacitance changes may be sensed by an ECG electrode and result in artifacts manifested as large amplitude signals within the ECG signal. These large amplitude signals may be mistaken for P or T waves of the ECG signal resulting in misinterpretation by the patient monitor.

The EMG electrical signals may interfere with ECG signals especially when a patient is moving or shivering. EMG interference may appear as narrow, frequent spikes within the ECG signal. Though filtering may be used to eliminate EMG interference to a considerable degree, occasional EMG spikes within the ECG signal may be mistaken for QRS-complexes. Noisy ECG signals in turn may result in an erroneous heart rate (HR) and other erroneous ECG derived parameters.

A noise-free ECG signal may reflect the rhythmic activity of the heart. This rhythm may be illustrated by a repeated PQRST pattern. The PQRST pattern may not be stationary, and exhibits variations due to physiological changes in heartbeat activity over time. However, these beat-to-beat variations may generally be minor over short windows of time, such as 4-5 beats. Over this short window of time, a pattern may exhibit certain features of the PQRST, such as: a) a silent period followed by a wide deflection, or P wave; b) another short silent period followed by a sudden small sharp deflection of a Q wave; c) a sharp, short duration and high amplitude R wave deflection; d) an S wave slightly larger than the Q wave that immediately follows the R wave; and/or e) a silent period followed by a relatively wide and small amplitude T wave. The time window of the PQRST pattern may include frequency components in the range of 0.5-40 Hz.

Artifacts that contaminate the ECG signals often have frequency components in the same range as the PQRST pattern. However, clinicians may visually distinguish the PQRST pattern from an artifact based upon distinct features, as explained above.

Conventional methods used to extract features from an ECG signal may be categorized into statistical methods, deterministic methods, or a combination of both. Statistical methods may produce statistical information from an ECG signal in the frequency domain or the time domain. Also, a wavelet decomposition of the ECG signal may be utilized as a technique for evaluation and to provide more effective time-frequency tradeoffs.

Conventional blind signal processing (BSP) algorithms have been utilized in biomedical engineering, medical imaging, and speech enhancement. BSP algorithms do not utilize a priori knowledge of the time domain characteristics of a signal, but rather depend on the knowledge of the statistical properties of the signal. Furthermore, BSP techniques do not use training data and may therefore be used in an unsupervised mode.

BSP techniques may include three major algorithms: a) Blind Signal Separation and Extraction (BSS/BSE); b) Independent Component Analysis (ICA); and c) Blind Multi-Channel Blind Deconvolution (MBD). These algorithms may rely on statistical information estimated online or in batch mode from a signal. For example, BSS/BSE techniques may separate a linear mixture of an unknown number of signals that are not completely statistically independent using second order statistics. Conversely, ICA techniques may utilize higher order statistics to separate statistically independent signals.

A conventional BSP algorithm has been utilized to separate an ECG signal from a noisy mixture of ECG signals. First, mixtures of synthesized signals were used to validate the algorithms. Next, the BSP algorithm was applied on the ECG signals recorded from three different leads attached to a single patient.

A conventional wavelet transform may provide a mechanism for multi-resolution analysis of a time domain signal. An output produced by the wavelet transform may be similar to an output of matched filters. Further, the output of the wavelet transform may be maximized when the filters match the signal. Thus a biorthognonal wavelet may produce a best match for the QRS complex of an ECG signal, since a biorthogonal wavelet may be very close in shape to a QRS complex. However, this same biorthogonal wavelet may not match P and T waves of the signal with acceptable; accuracy. The conventional single wavelet basis function may not be flexible enough to represent a complicated non-stationary signal such as the ECG signal.

Conventional orthonormal wavelet bases generally have compact support. A dictionary of pre-defined scaling functions may be available for use in the matching process. The matching algorithm may select the suitable scaling function from the dictionary for providing the best match to the signal. Selection of the scaling function may result in optimal matching for the lower band of the signal. However, using a dictionary of wavelets to optimize a match may be influenced by the contents of the dictionary. A dictionary of predefined functions may not include the functions needed to produce the best match for a particular signal. One conventional algorithm has addressed this problem with a specifically designed wavelet and corresponding scaling function for matching a signal. This conventional algorithm is based on multi-resolution analysis (MRA) to develop an orthonormal wavelet that matches a given signal.

### SUMMARY OF THE INVENTION

A system in accordance with the present invention eliminates artifacts from an electrocardiogram signal. The system includes a monitor for receiving an electrocardiogram signal from a patient and a microprocessor utilizing a shift-invariant wavelet transform for decomposing the electrocardiogram signal into a plurality of scales. The microprocessor applies rules to the scales for removing artifacts from the scales. The microprocessor reassembles the plurality of scales to produce a time domain waveform that is an accurate electrocardiogram signal without the artifacts.

A method in accordance with the present invention eliminates artifacts from an electrocardiogram signal. The method comprises the steps of receiving an electrocardiogram signal from a patient; utilizing a shift-invariant wavelet transform for decomposing the electrocardiogram signal into a plurality of scales; applying rules to the scales for removing artifacts from the scales; reassembling the plurality of scales to produce a time domain waveform that is an accurate electrocardiogram signal without the artifacts.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a schematic representation of a decomposition of an example signal;

FIG. 2 is a schematic representation of a decomposition of an example ECG signal;

FIG. 3 is a schematic representation of an example dual-tree complex wavelet;

FIG. 4 is a schematic representation of a decomposition of an example ideal artifact-free ECG signal;

FIG. 5 is a schematic representation of a decomposition of an example actual artifact-free signal;

FIG. 6 is a table of energy values for each scale in FIG. 5;

FIG. 7 is a schematic representation of a comparison of part of the FIG. 5 and a reconstructed part of FIG. 5;

FIG. 8 is a schematic representation of a DTCWT decomposition of an ECG signal with an example artifact;

FIG. 9 is a schematic representation of a DTCWT decomposition of an ECG signal with another example artifact;

FIG. 10 is a schematic representation of a comparison of part of an example signal and a reconstructed part of that signal;

FIG. 11 is a schematic representation of a comparison of parts of two example signals and reconstructed parts of those signals;

FIG. 12 is a schematic representation of a comparison of parts of two example signals and reconstructed parts of those signals;

FIG. 13 is a schematic representation of a comparison of parts of two example signals and reconstructed parts of those signals;

FIG. 14 is a schematic representation of a comparison of parts of two example signals and: reconstructed parts of those signals;

FIG. 15 is a schematic representation of a comparison of part of another example signal and a reconstructed part of that signal; and

FIG. 16 is a table comparing various ECG signal processing methods.

FIG. 17 is a schematic representation of a system in accordance with the present invention.

FIG. 18 is a schematic representation of a method in accordance with the present invention.

### DESCRIPTION OF AN EXAMPLE EMBODIMENT

A Discrete Wavelet Transform (DWT) may provide an efficient representation of an ECG signal containing singularities, lacks periodicity, and is non-stationary. Such a signal is typically not well represented by a periodic sinusoidal collection of basis functions as used in a conventional Fourier transform. The DWT may replace a collection of infinitely oscillating complex sinusoidal basis functions of a Fourier transform with a set of basis wavelets each having compact support. The DWT basis function may be a shifted and dilated version of a fundamental real-valued band-pass wavelet combined with a shifted version of a real-valued low-pass scaling function.

The wavelet and scaling function of the DWT may provide an orthonormal basis system similar to the orthonormal complex sinusoidal basis functions of the Fourier transform. However, the DWT lacks the translation (shift) invariance property of a Fourier transform. This property of the DWT is the result of the fact that the wavelet bases of a DWT are real-valued, whereas the bases of a Fourier transform are complex-valued sinusoidal functions.

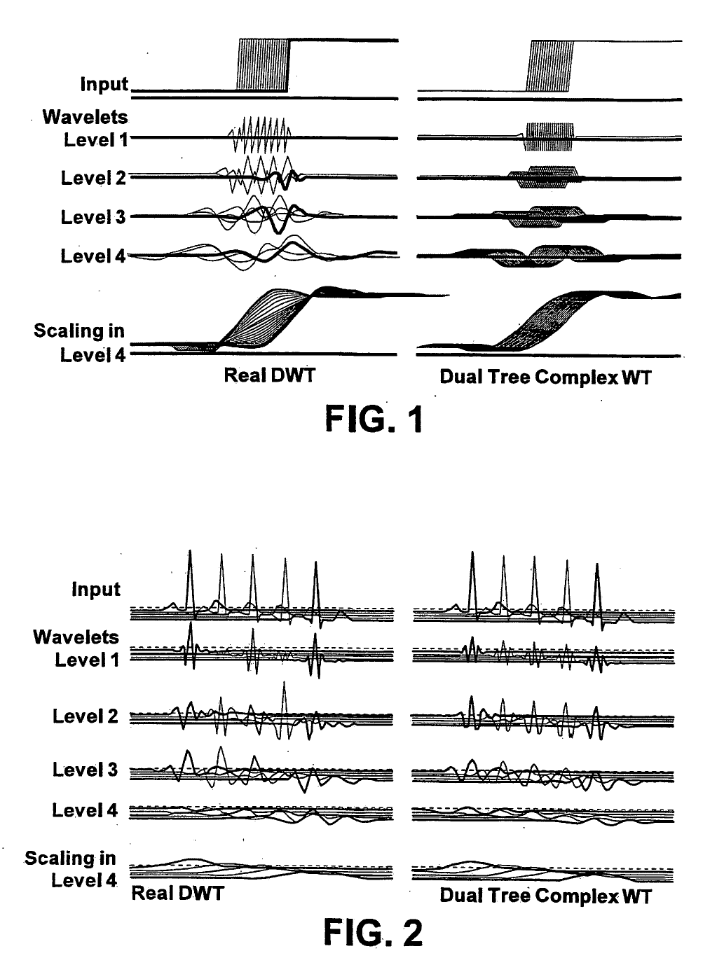

A system in accordance with the present invention may utilize a Dual-Tree Complex Discrete Wavelet Transform (DTCWT) to overcome the shift-variance issue of the DWT. To demonstrate the shift-variance of the DWT as shown in FIG. 1, a unit step function may be decomposed to four scales using a DWT and a DTCWT. The unit, step function has been shifted one sample to the right and decomposed by the DWT and the DTCWT. The shifting/decomposition process was repeated 16 times and the decompositions are shown in FIG. 1. The DWT produced different decomposition of the shifted unit step function at each scale.

As shown in FIG. 2, the same process was applied on 16 beats that were created from a single ECG signal. The 16 beats were shifted in time by a single sample and decomposed using the DWT and the DTCWT. The DWT decomposed the ECG signal into scales that included different energy amounts for the shifted ECG signal. This difference in the captured energy is considerable and provides an inconsistent decomposition of the ECG signal. Such inconsistency can impair the applicability of the DWT to situations where the ECG signal is corrupted by artifacts. FIG. 2 illustrates this difference in the amount of energy in the various scales of the DWT decomposition. Please note that only five beats were plotted in FIG. 2, for clarity.

The Dual-Tree Complex Wavelet may generate discrete complex wavelets. Generally, a discrete complex wavelet bases may be generated with either non-redundant bases or redundant bases. Non-redundant bases produce orthonormal or biorthogonal wavelets. A rigid constraint associated with an orthonormal or biorthogonal wavelet basis results in four issues: a) the filter coefficients are oscillatory in the neighborhood of the singularities; b) translation (shift) variance; c) aliasing; and d) lack of directionality for image processing. These difficulties may be overcome by a redundant generation method, which may slightly increase computational complexity associated with the decomposition.

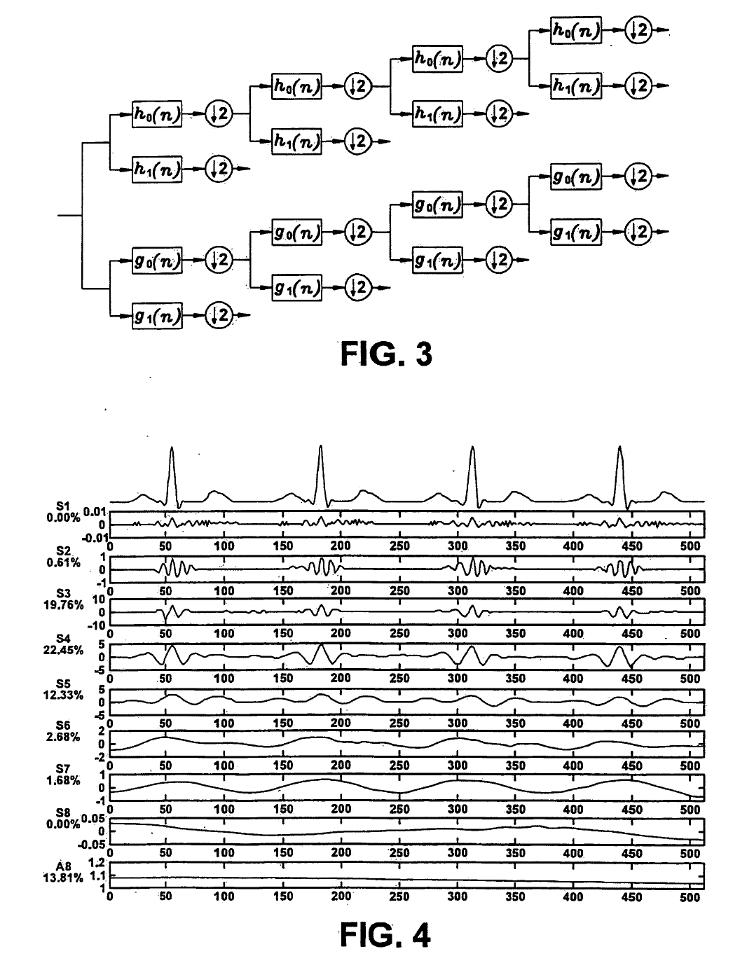

A DTCWT may be based on two filter bank trees. The two filter bank trees may represent real-valued filters where the first bank, or “h” filters, represents a real part and the second bank, or “g” filters, represents an imaginary part. The two filter banks may be separated such that their computations do not depend on one another. FIG. 3 shows a schematic representation of the two filter banks.

The “g” filters of the second bank may be obtained from the “h” filters of the first bank using a Hilbert transform. As a result, the support of the “g” filters may be undesirably infinite. Thus, finitely supported “g” filters may be designed to approximate the infinitely supported “g” filters, derived by Hilbert transformation. That is:

$$where \text{:}$$

$${Ψ}_{g} ( t ) \approx H \{ {Ψ}_{h} ( t ) \}$$

$${ψ}_{h} ( t ) = \sqrt{2} {\sum }_{n}^{ } {h}_{1} ( n ) {ϕ}_{h} ( t ) , \\ {ϕ}_{h} ( t ) = \sqrt{2} {\sum }_{n}^{ } {h}_{0} ( n ) {ϕ}_{h} ( t ) , \\ {h}_{1} ( n ) = {( - 1 )}^{n} {h}_{0} ( d - n )$$

$$and$$

$${ψ}_{g} ( t ) = \sqrt{2} {\sum }_{n}^{ } {g}_{1} ( n ) {ϕ}_{g} ( t ) , \\ {ϕ}_{g} ( t ) = \sqrt{2} {\sum }_{n}^{ } {g}_{0} ( n ) {ϕ}_{g} ( t ) , \\ {g}_{1} ( n ) = {( - 1 )}^{n} {g}_{0} ( d - n ) .$$

The filters h₀ should be shifter from g₀ by approximately half-sample. That is:

g₀(n)≈h₀(n−0.5) [symbol] Ψ_{g}(t)≈H{Ψₕ(t)}.

The half-sample delay between g₀ and h₀ may be equivalent to uniform over-sampling of the low-pass signal at each scale by 2:1. Therefore, aliasing that may be generated by down-sampling occurring at each scale may be avoided.

The DTCWT provides a shift-invariant decomposition of the ECG signal. This consistent decomposition of the ECG signal may thus establish a reliable method for reducing and/or eliminating artifacts in an ECG signal.

An ECG signal may include both artifact-free segments and artifactual segments. Therefore, an ECG signal may be decomposed into a given set of scales (8, for example) and the energy levels of these (8) scales may be analyzed. In order to compare the energy amounts captured by each scale, summations of squared values of the components of each scale may be calculated. The energy of each scale may be normalized by dividing this energy by the total energy in the ECG signal. That is:

$${E}_{{s}_{n}} = {\sum }_{i}^{N} {S}_{n}^{2} ( i )$$

$${P}_{n} = \frac{{E}_{{s}_{n}}}{{\sum }_{i}^{N} {S}_{ECG}^{2} ( i )}$$

where Sₙ(i)=component i in scale n,

N=number of components in Scale n,

Pₙ=E_{Sn} normalized,

S_{ECG}(i)=sample i of the ECG signal.

The normalized value of Eₛₙ, Pₙ, may be used to compare the amount of energy captured at each scale, or the energy percentage at each scale.

The energy composition of the ECG signal may be studied by applying the DTCWT to an ideal ECG signal. The ideal ECG signal may be synthesized by concatenating five ideal beats to form a single signal. The decomposition of the ideal ECG signal may include a number of scales (8, for example) and an approximation function. The energy percentage at each scale may then be calculated. FIG. 4 shows an example of the ideal ECG signal decomposed at each of the 8 scales, the approximation function, and the energy percentage for each scale. Each scale has the same composition for each beat of the ECG signal. The majority of the signal energy is contained in scales S3 to S7 and the approximation function. Most of the QRS-complex energy is contained in the scales S3 and S4 and most of the P-wave and the T-wave energy is contained in scales S5 and S6. Of, course fewer and more scales can be used within the same method of analysis.

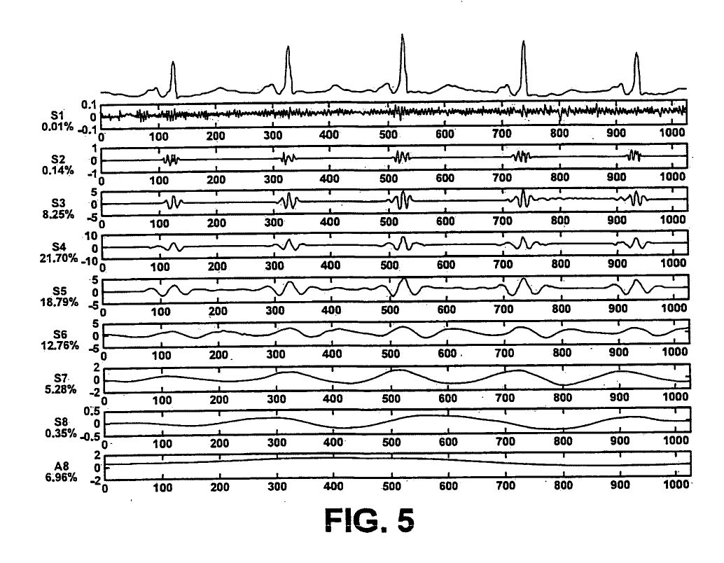

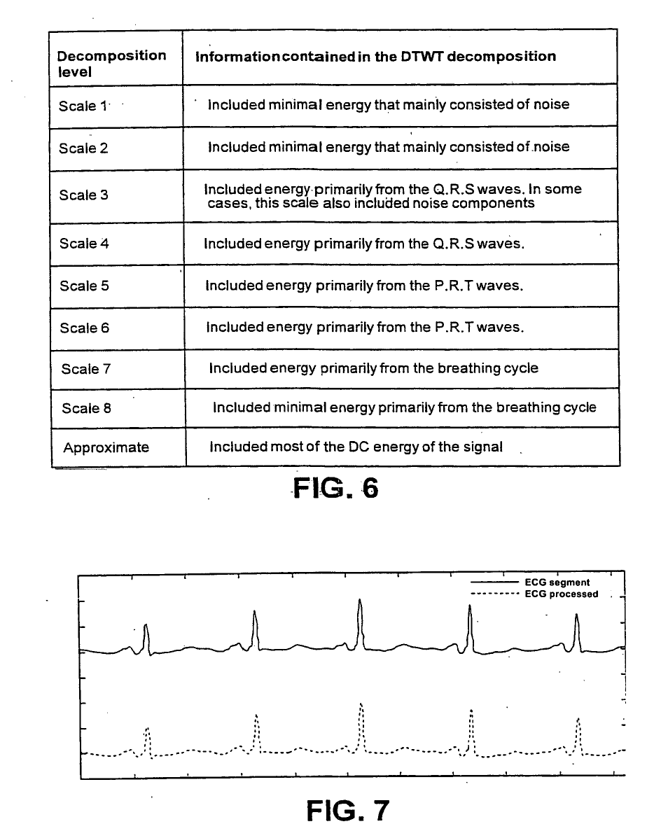

In FIG. 5, the DTCWT was applied to an ECG signal acquired from an actual patient in an operating room. Like the ideal ECG signal of FIG. 4, most of the ECG energy was contained in scales S3 to S7. FIG. 6 summarizes the energy contained in that ECG signal at each scale.

This ECG signal was reconstructed using scales S3 to S7. As shown in FIG. 7, this reconstructed ECG signal is very close to the original ECG signal.

Thus, the DTCWT provided a consistent decomposition of an ECG signal without artifacts. The effect of an artifact on the DTCWT decomposition is described below. In particular, the utility of the DTCWT for artifact detection, removal, and ECG reconstruction is examined.

ECG signals contain frequency content over a wide range, for example 0.5 to 40 Hertz. Sources for artifacts that may contaminate an ECG signal in the operating room may also include signal content in the same frequency range as the ECG. Consequently, separating the artifact signal from the ECG signal may be a difficult and challenging problem, and conventional linear frequency domain filtering is not a viable option. The energy decomposition over the DTCWT scales has been studied for two types of artifacts. First, artifacts that contaminate the high frequency components of the ECG signal that may influence the ability to detect and quantify tachycardia signals. Second, artifacts that contaminate the low frequency components of the ECG signal that may influence the ability to detect and quantify bradycardia signals. Consideration of these two types of artifacts ensures that eliminating these artifacts won't alter the underlying cardiac arrhythmias such as bradycardia and tachycardia.

An example of a high frequency artifact source is an electrosurgery unit that contaminates high frequency scales. An example of a low frequency artifact source is stretching of electrode pads, typically generating high-energy components at low frequencies. Attempts at eliminating these artifacts could result in removing tachycardia or bradycardia components as well as distorting other features such as ST segment from a measured ECG signal. An ECG signal containing incidents of these types of artifacts have been decomposed and analyzed below to determine scales with the higher amounts of energy associated with these two types Of artifacts.

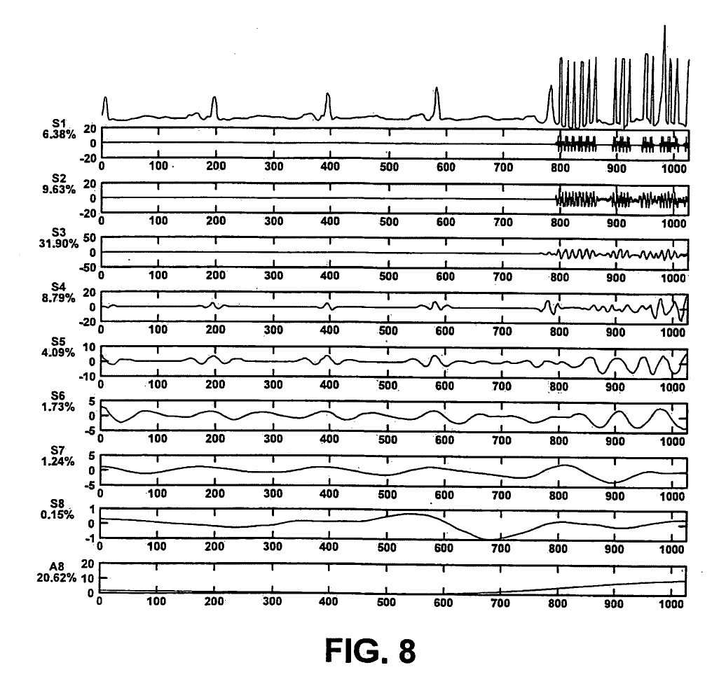

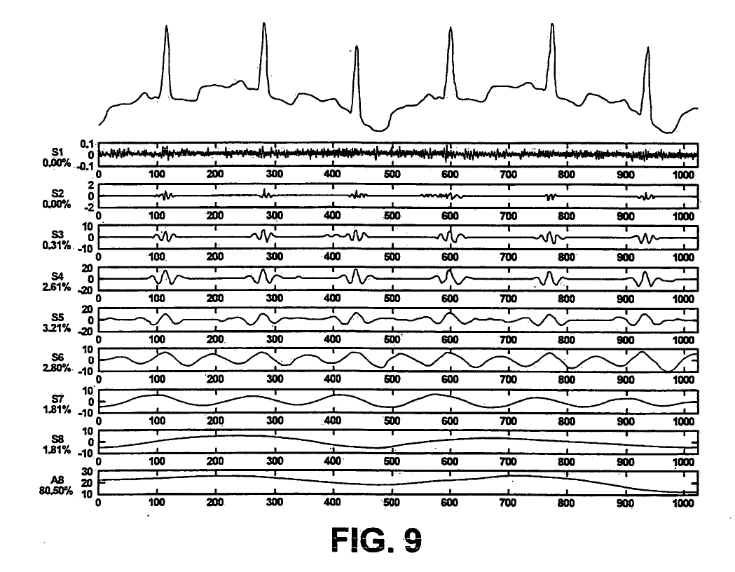

Analysis of a DTCWT decomposed ECG signal contaminated with an artifact from an electrosurgical unit reveals that scales 1 and 2 contain most of the artifact energy. However, scales 3 and 4 also contain quite an amount of the artifact energy that may mask the energy Of the QRS complex. Therefore, techniques will be needed to carefully eliminate the artifact energy from scales 3 and 4 without impacting the QRS complex or the tachycardia. FIG. 8 shows a decomposition of the contaminated ECG signal and the energy percentage captured at each scale.

The low frequency artifact is contained in scale 8 and in the approximation signal. Scale 8 and the approximation signal include the majority of the energy due to wandering in the signal baseline and the DC gain of the ECG signal. The P-wave and the T-wave are unaffected since the P-wave and the T-wave are mainly contained in scales 5 and 6. FIG. 9 shows a low frequency artifact and its decomposition.

A system in accordance with the present invention uses a DTCWT to decompose an ECG signal into scales for consistently capturing the percentages of the signal's energy that include the QRS complex, the P-wave, the T-wave, as well as the various artifacts that are to be eliminated from the ECG signal. The consistency of the decomposition facilitates establishment of rules that may be used to de-noise each scale prior to signal reconstruction.

Rules to de-noise the scales may be inferred from the energy levels captured at each scale. As stated above, the scales of the decompositions may include different levels of energy from the ECG signal. In a particular embodiment of the invention that included an 8-scale signal decomposition, these scales may be classified into four main categories that may be de-noised by different mechanisms. These four categories are:
1) Scales 1 and 2 include high frequency signal components with low amplitude. These scales do not contribute to the ECG signal, but rather to noise. Dropping these scales may reduce noise in the ECG signal.
2) Scales 3 and 4 include most of the energy in the QRS complex. However, there is some interference with the high frequency signal. A clamping rule may be used to eliminate energy samples that are much higher than the neighboring samples.
3) Scales 5, 6, and 7 include energy associated with the P-wave, the R-wave, and the T-wave. These scales do not seem to be susceptible to noise, and no rule was necessary to de-noise them.
4) Scale 8 and the approximation function include some energy from wandering of the signal base line and the DC components, and hence these may be disregarded.

These four rules may be applied to an ECG signal to reduce the noise. However, in applying these de-noising rules, some useful information in the ECG signal may also be eliminated. For example, dropping or clamping the high-energy scales may alter the tachycardia segments of the ECG beats. Also, disregarding the signals in the low-energy scale may eliminate segments containing arrhythmias or bradycardia. Therefore, careful examination of the original ECG signals verses the processed signals was essential and it has indicated that no useful information was altered or lost in the de-noising and subsequent reconstruction process.

The morphology of an ECG signal may differ from patient to patient, as well as within the chronological history of any given patient. Because the various ECG signal morphologies that are of interest and importance may be impacted differently due to the application of the de-noising rules, a verification may assess the impact of the de-noising rules for the same subject over time and also for different subjects.

ECG signals have been acquired from seven patients who underwent cardiac surgery (IRB approved). The ECG signals were acquired by GE Marquette® Solar 9500 patient monitors for an average of four hours for each patient and at a sample rate of 120 Hz. Identification information was removed to ensure confidentiality. The ECG signals were processed with the same set of de-noising rules without adapting any of the rules to a specific patient.

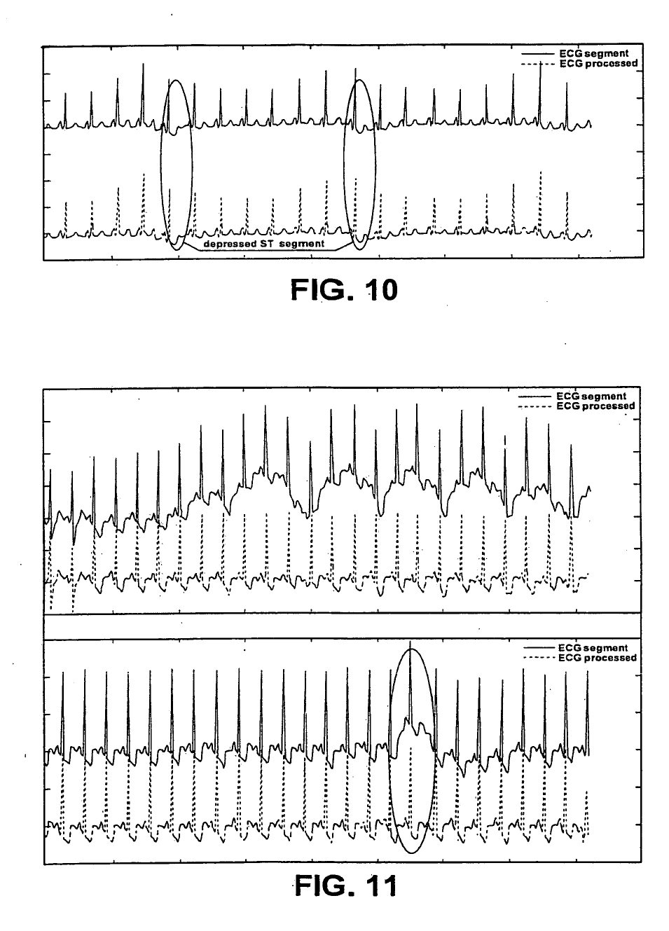

The processed ECG signals were verified versus the original ones to ensure that no alteration occurred to useful information, such as tachycardia, bradycardia, and arrhythmia. The ECG signals included various types of artifacts, such as a wandering of the signal baseline, generic low frequency artifacts, corruption due to a electrosurgical unit, and generic high frequency artifacts. Samples of artifactual ECG signals and their corresponding corrected signals are shown in FIGS. 10-15.

The wander of the signal baseline was removed as displayed in FIG. 10. Eliminating the wandering baseline did not have any affect on the depressed ST segments.

Artifacts that consist of low frequency components were removed without altering the P-wave, or T-waves. Also, FIG. 11 shows artifact elimination without altering the tachycardia or varying the elongated ECG signals.

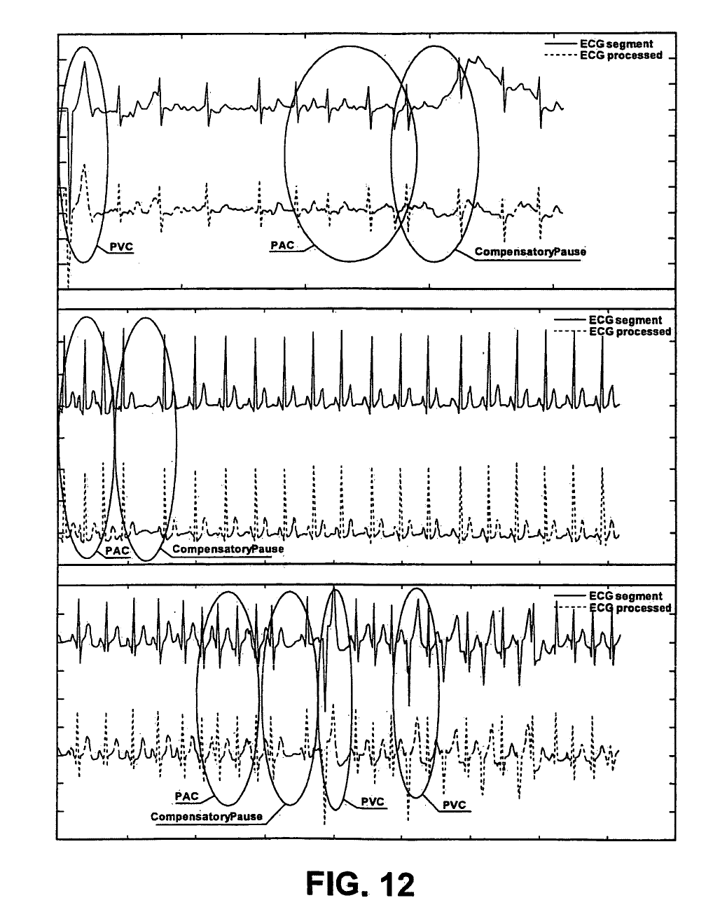

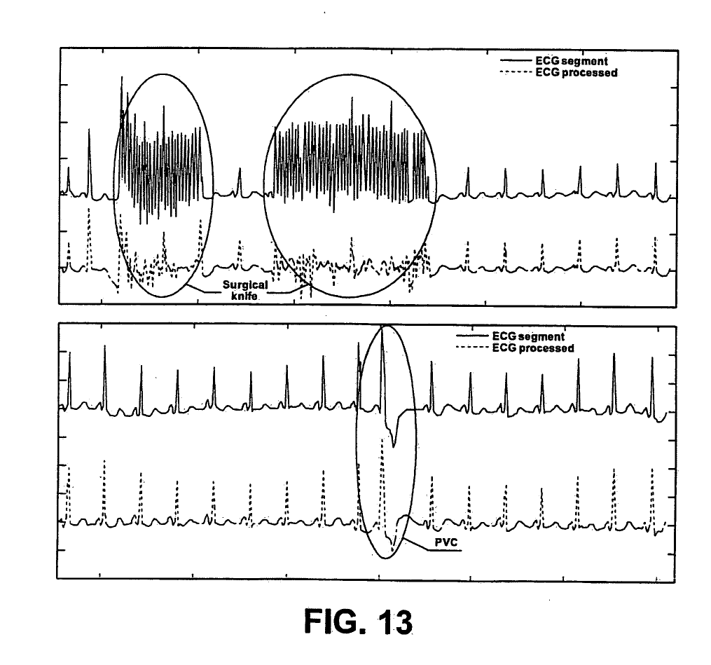

Premature ventricular contraction, premature atrial contraction, and compensating pause were preserved, as shown by three samples of these arrhythmias in FIG. 12. A sample of a electrosurgical unit artifact is shown in the upper graph of FIG. 13. The ability to eliminate this artifact is limited due to the high energy included in this artifact that may mask the original ECG signal. However, reducing the impact of this artifact on the ECG signal did not impact the premature ventricular contraction displayed in the lower graph of FIG. 13.

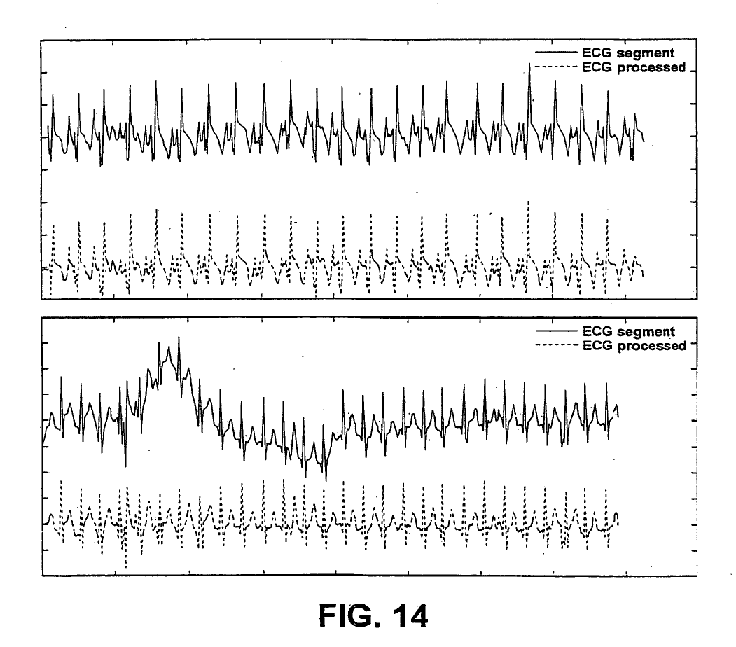

One ECG signal out of the seven had different morphology than the usual due to a pacemaker. In spite of this, the signal was well preserved even after processing. The paced ECG signal and its processing are shown in the upper graph of FIG. 14. Further, low frequency artifacts were removed from the paced ECG signal without altering the original signal. The corrected paced signal is shown in the lower graph of FIG. 14.

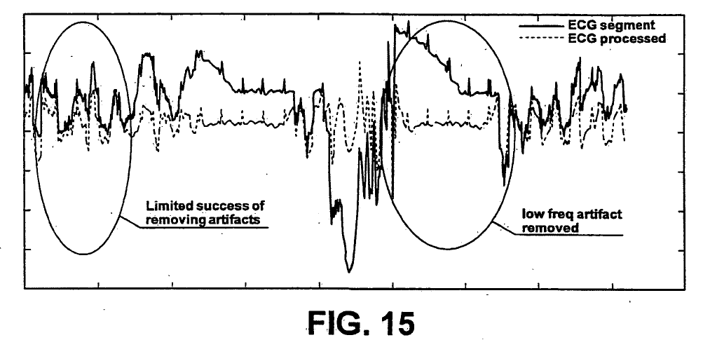

The four de-noising rules eliminated the low frequency artifacts. Also, the rules, at least partially, eliminated the large amount of energy introduced by an electrosurgical unit. The electrosurgical unit is an example of an artifact that may destroy an ECG signal. In the case of a completely distorted ECG signal, the de-noising rules may have limited ability to reconstruct an ECG signal, as shown in FIG. 15.

An ECG signal collected in an operating room and intensive care unit may be susceptible to many sources of artifacts. The artifacts may distort the hemodynamic signals displayed by a patient monitor, which could in turn lead to an incorrect interpretation of clinical information and generation of false alarms. These undesirable effects of artifacts may compromise patient care and safety.

Artifacts in an ECG signal have been rigorously studied and various conventional techniques have been proposed to detect and eliminate them. Adaptive filtering was one of the first conventional techniques used to address the time varying nature of interfering noise. Neural networks were later proposed to provide nonlinear adaptation of filters. The wavelet method has also been used to detect the P and T waves, as well as the QRS complex, in a noisy ECG signal. Further, Independent Component Analysis (ICA) has been: used to eliminate abrupt noise, as well as continuous noise. These conventional techniques have been generally applied to ECG signals acquired from environments other than an operating room. Further, each of these conventional techniques focused on a single aspect of the ECG signal or a specific type of artifact.

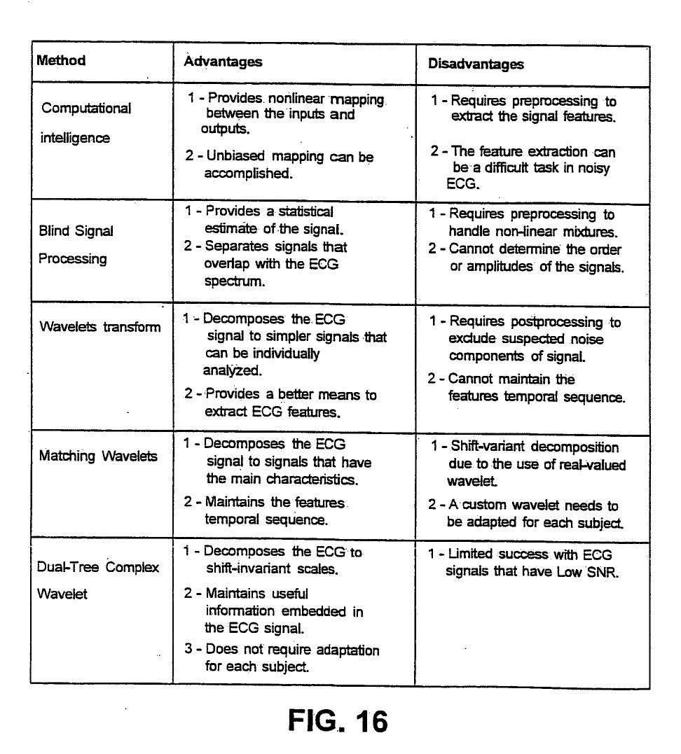

Consequently, conventional techniques may be incapable of processing a variety of ECG signal types and artifacts, commonly encountered in an operating room. Thus, a system in accordance with the present invention utilizes a dual-tree complex wavelet for providing a shift-invariant decomposition of the ECG signals. The decomposition generated by the system is adequately consistent and may be used to generate a set of rules for eliminating certain types of artifacts without altering the base ECG signal or removing cardiac arrhythmias. Computational intelligence, blind signal processing, wavelet transforms, matching wavelets, and dual-tree complex wavelets are compared in FIG. 16.

Thus, a system in accordance with the present invention may utilize dual-tree complex wavelet transforms to eliminate artifacts from an ECG signal while still maintaining the temporal structure of the ECG signal and decomposing the ECG signal into various scales. Further, the complex-valued dual-tree complex wavelet provides a shift-invariant decomposition. Shift-invariant decompositions may provide a consistent means for further analysis of the ECG signal. This further analysis may eliminate artifacts and improve alerts generated by patient monitors in an operating room.

The consistent decomposition of the ECG signal provided by the system allows for effective elimination of ECG artifacts. The System may employ rules for eliminating destructive noise, such as the electrosurgical unit. Neural networks may be used to determine artifact rejection rules. For instance, a neural network may be used to develop more complex rules that combine more than a single scale in an attempt to eliminate artifacts with greater accuracy.

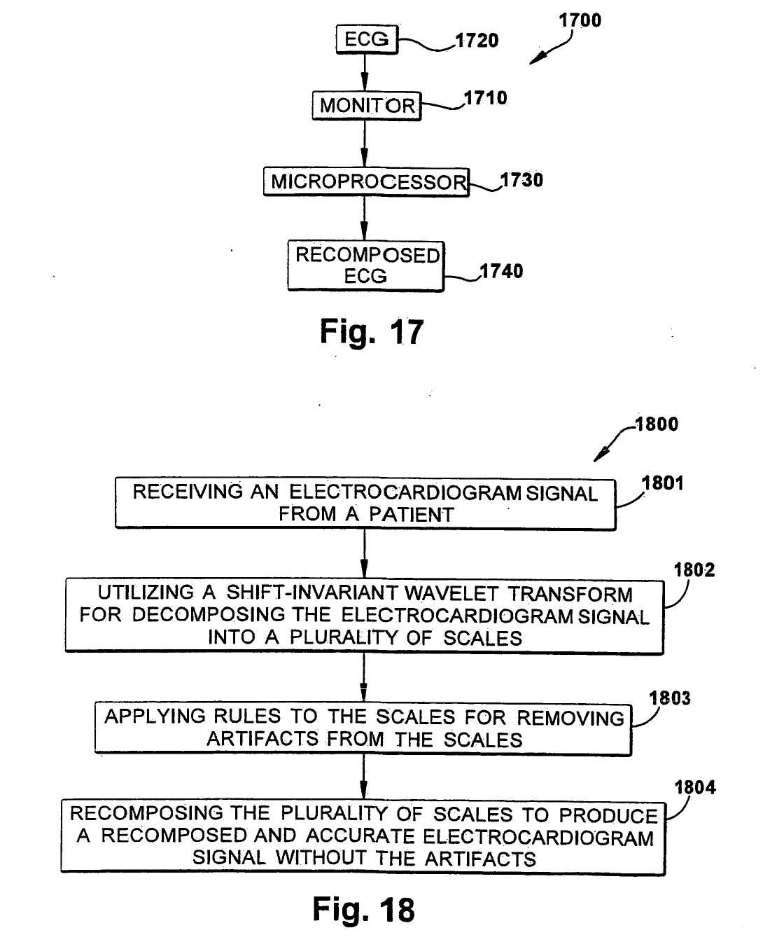

As shown in FIG. 17, a system 1700 in accordance with the present invention eliminates artifacts from an electrocardiogram signal. The system 1700 includes a monitor 1710 for receiving an electrocardiogram signal 1720 from a patient and a microprocessor 1730 utilizing a shift-invariant wavelet transform for decomposing the electrocardiogram signal into a plurality of scales. The microprocessor 1730 applies rules to the scales for removing artifacts from the scales. The microprocessor 1730 reassembles the plurality of scales to produce a reconstructed and accurate electrocardiogram signal 1740 without the artifacts. The monitor 1710 and the microprocessor 1730 may be contained within the same device.

As shown in FIG. 18, a method 1800 in accordance with the present invention eliminates artifacts from an electrocardiogram signal. The method 1800 comprises the steps of: receiving 1801 an electrocardiogram signal from a patient; utilizing 1802 a shift-invariant wavelet transform for decomposing the electrocardiogram signal into a plurality of scales; applying 1803 rules to the scales for removing artifacts from the scales; reassembling 1804 the plurality of scales to produce a reconstructed and accurate electrocardiogram signal without the artifacts.

In order to provide a context for the various aspects of the present invention, the following discussion is intended to provide a brief, general description of a suitable computing environment in which the various aspects of the present invention may be implemented. While the invention has been described above in the general context of computer-executable instructions of a computer program that runs on a computer, those skilled in the art will recognize that the invention also may be implemented in combination with other program modules.

Generally, program modules include routines, programs, components, data structures, etc. that perform particular tasks or implement particular abstract data types. Moreover, those skilled in the art will appreciate that the inventive methods may be practiced with other computer system configurations, including single-processor or multiprocessor computer systems, minicomputers, mainframe computers, as well as personal computers, hand-held computing devices, microprocessor-based or programmable consumer electronics, and the like. The illustrated aspects of the invention may also be practiced in distributed computing environments where tasks are performed by remote processing devices that are linked through a communications argument model. However, some, if not all aspects of the invention can be practiced on stand-alone computers. In a distributed computing environment, program modules may be located in both local and remote memory storage devices.

An exemplary system for implementing the various aspects of the invention includes a conventional server computer, including a processing unit, a system memory, and a system bus that couples various system components including the system memory to the processing unit. The processing unit may be any of various commercially available processors. Dual microprocessors and other multi-processor architectures also can be used as the processing unit. The system bus may be any of several types of bus structure including a memory bus or memory controller, a peripheral bus, and a local bus using any of a variety of conventional bus architectures. The system memory includes read only memory (ROM) and random access memory (RAM). A basic input/output system (BIOS), containing the basic routines that help to transfer information between elements within the server computer, such as during start-up, is stored in ROM.

The server computer further includes a hard disk drive, a magnetic disk drive, e.g., to read from or write to a removable disk, and an optical disk drive, e.g., for reading a CD-ROM disk or to read from or write to other optical media. The hard disk drive, magnetic disk drive, and optical disk drive are connected to the system bus by a hard disk drive interface, a magnetic disk drive interface, and an optical drive interface, respectively. The drives and their associated computer-readable media provide nonvolatile storage of data, data structures, computer-executable instructions, etc., for the server computer. Although the description of computer-readable media above refers to a hard disk, a removable magnetic disk and a CD, it should be appreciated by those skilled in the art that other types of media which are readable by a computer, such as magnetic cassettes, flash memory cards; digital video disks, Bernoulli cartridges, and the like, may also be used in the exemplary operating environment, and further that any such media may contain computer-executable instructions for performing the methods of the present invention.

A number of program modules may be stored in the drives and RAM, including an operating system, one or more application programs, other program modules, and program data. A user may enter commands and information into the server computer through a keyboard and a pointing device, such as a mouse. Other input devices (not shown) may include a microphone, a joystick, a game pad, a satellite dish, a scanner, or the like. These and other input devices are often connected to the processing unit through a serial port interface that is coupled to the system bus, but may be connected by other interfaces, such as a parallel port, a game port or a universal serial bus (USB). A monitor or other type of display device is also connected to the system bus via an interface, such as a video adapter. In addition to the monitor, computers typically include other peripheral output devices (not shown), such as speaker and printers.

The server computer may operate in a networked environment using logical connections to one or more remote computers, such as a remote client computer. The remote computer may be a workstation, a server computer, a router, a peer device or other common network node, and typically includes many or all of the elements described relative to the server computer. The logical connections include a local area network (LAN) and a wide area network (WAN). Such networking environments are commonplace in offices, enterprise-wide computer networks, intranets and the internet.

When used in a LAN networking environment, the server computer is connected to the local network through a network interface or adapter. When used in a WAN networking environment, the server computer typically includes a modem, or is connected to a communications server on the LAN, or has other means for establishing communications over the wide area network, such as the internet. The modem, which may be internal or external, is connected to the system bus via the serial port interface. In a networked environment, program modules depicted relative to the server computer, or portions thereof, may be stored in the remote memory storage device. It will be appreciated that the network connections shown are exemplary and other means of establishing a communications link between the computers may be used.

In accordance with the practices of persons skilled in the art of computer programming, the present invention has been described with reference to acts and symbolic representations of operations that are performed by a computer, such as the server computer, unless otherwise indicated. Such acts and operations are sometimes referred to as being computer-executed. It will be appreciated that the acts and symbolically represented operations include the manipulation by the processing unit of electrical signals representing data bits which causes a resulting transformation or reduction of the electrical signal representation, and the maintenance of data bits at memory locations in the memory system (including the system memory, hard drive, floppy disks, and CD-ROM) to thereby reconfigure or otherwise alter the computer system's operation, as well as other processing of signals. The memory locations where such data bits are maintained are physical locations that have particular electrical, magnetic, or optical properties corresponding to the data bits.

While there is shown and described herein certain specific alternative forms of the invention, it will be readily apparent to those skilled in the art that the invention is not so limited, but is; susceptible to various modifications and rearrangements in design and materials without departing from the spirit and scope of the invention. In particular, it should be noted that the present invention is subject to modification with regard to the dimensional relationships and parameters set forth herein and modifications in assembly, materials, size, shape, and use.

From the above description of the invention, those skilled in the art will perceive improvements, changes and modifications. Such improvements, changes and modifications within the skill of the art are intended to be covered by the appended claims.

Having described the invention, the following is claimed:

1. A system for eliminating artifacts from an electrocardiogram signal, said system comprising:
a monitor for receiving an electrocardiogram signal from a patient; and
a microprocessor utilizing a shift-invariant wavelet transform for decomposing the electrocardiogram signal into a plurality of scales, said microprocessor applying rules to the scales for removing artifacts from the scales, said microprocessor reassembling the plurality of scales to produce a reconstructed and accurate electrocardiogram signal without the artifacts.

2. The system as set forth in claim 1 wherein said microprocessor applies a first rule to scales 1 and 2 of the plurality of scales for removing high frequency artifacts from scales 1 and 2 to produce a reconstructed electrocardiogram signal without high frequency artifacts.

3. The system as set forth in claim 2 wherein said microprocessor applies a second rule to scales 3 and 4 of the plurality of scales for removing high-energy artifacts from scales 3 and 4 to produce a reconstructed electrocardiogram signal without high-energy artifacts.

4. The system as set forth in claim 3 wherein said microprocessor applies a third rule to scales 5, 6, and 7 of the plurality of scales for removing artifacts from scales 5, 6, and 7 to produce a reconstructed electrocardiogram signal without artifacts.

5. The system as set forth in claim 4 wherein the third rule comprises no rule.

6. The system as set forth in claim 4 wherein said microprocessor applies a fourth rule to scale 8 of the plurality of scales for removing a wandering baseline artifact from scale 8 to produce a reconstructed electrocardiogram signal without a wandering baseline artifact.

7. The system as set forth in claim 1 wherein the shift-invariant wavelet transform is a dual-tree complex wavelet transform.

8. The system as set forth in claim 1 wherein said microprocessor normalizes energy in each of the plurality of scales for comparing the amount of energy in each of the plurality of scales.

9. The system as set forth in claim 1 wherein the reconstructed electrocardiogram signal includes tachycardia, bradycardia, and arrhythmia segments preserved from the electrocardiogram signal.

10. The system as set forth in claim 1 wherein the reconstructed electrocardiogram signal includes premature ventricular contraction, premature atrial contraction, and compensating pause segments preserved from the electrocardiogram signal.

11. A method for eliminating artifacts from an electrocardiogram signal, said method comprising the steps of:
receiving an electrocardiogram signal from a patient;
utilizing a shift-invariant wavelet transform for decomposing the electrocardiogram signal into a plurality of scales;
applying rules to the scales for removing artifacts from the scales; and
reassembling the plurality of scales to reconstruct an accurate electrocardiogram signal without the artifacts.

12. The method as set forth in claim 11 further including the step of applying a first rule to scales 1 and 2 of the plurality of scales for removing high frequency artifacts from scales 1 and 2 to reconstruct electrocardiogram signal without high frequency artifacts.

13. The method as set forth in claim 12 further including the step of applying a second rule to scales 3 and 4 of the plurality of scales for removing high-energy artifacts from scales 3 and 4 to reconstruct electrocardiogram signal without high-energy artifacts.

14. The method as set forth in claim 13 further including the step of applying a third rule to scales 5, 6, and 7 of the plurality of scales for removing artifacts from scales 5, 6, and 7 to reconstruct electrocardiogram signal without artifacts.

15. The method as set forth in claim 14 wherein the third rule comprises no rule.

16. The method as set forth in claim 15 further including the step of applying a fourth rule to scale 8 of the plurality of scales for removing a wandering baseline artifact from scale 8 to reconstruct electrocardiogram signal without a wandering baseline artifact.

17. The method as set forth in claim 11 wherein the shift-invariant wavelet transform is a dual-tree complex wavelet transform.

18. The method as set forth in claim 11 further including the step of normalizing energy in each of the plurality of scales for comparing the amount of energy in each of the plurality of scales.

19. The method as set forth in claim 11 wherein the reconstructed electrocardiogram signal includes tachycardia, bradycardia, and arrhythmia segments preserved from the electrocardiogram signal.

20. The method as set forth in claim 11 wherein the reconstructed electrocardiogram signal includes premature ventricular contraction, premature atrial contraction, and compensating pause segments preserved from the electrocardiogram signal.
